(19) United States
(12) Patent Application Publication
(10) Pub. No.: US 2009/0259249 A1
(43) Pub. Date: Oct. 15, 2009

(54) ARTERIOTOMY STAPLING SYSTEM FOR NON-ORTHOGONAL TISSUE TRACKS AND METHODS OF USE THEREIN

(75) Inventor: Richard D. Lobello, Johnston, RI (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/100,532

(22) Filed: Apr. 10, 2008

(51) Int. Cl. A61B 17/064; A61B 17/068

(52) U.S. Cl. 606/219; 227/175.1

(57) ABSTRACT

A stapling system for closing an arteriotomy includes a staple having four legs and a stapler that accommodates a non-orthogonal angle formed between a percutaneous tissue track and an arteriotomy. The staple is formed from a resilient material and is biased towards a static configuration in which a proximal portion of the staple is generally orthogonal to an imaginary axis of the stapling system. The stapler deforms the staple into a delivery configuration in which the pointed tips of the staple are staggered with respect to the axis of the stapling system. Once the staple is engaged in vessel tissue as desired about the arteriotomy, the staple is released from the stapler and tends to revert back to the static configuration in order to hold the arteriotomy closed. Methods of using the stapling system are also disclosed.

### FIELD OF THE INVENTION

The invention is generally directed to a medical stapling system for delivering a medical staple to a puncture arteriotomy.

### BACKGROUND OF THE INVENTION

Various cardiovascular procedures, such as angioplasty, stent placement and atherectomy, require inserting into and manipulating within the vasculature, medical guidewires and catheters adapted to perform those procedures. Access to the vasculature typically is through the femoral artery and is percutaneous, involving insertion of a needle in the region of the groin to form a track through subcutaneous tissue and to puncture and create an arteriotomy in the femoral artery. A guidewire is then advanced through the needle and into the femoral artery. The needle then is removed. An introducer sheath is then advanced over the guidewire, along the track and into the femoral artery. The sheath provides access into the femoral artery, through the arteriotomy, for guidewires, catheters or other instrumentalities in order to perform the selected procedure.

After the procedure has been completed, the procedural devices are removed and the arteriotomy must be closed. The size of the puncture: opening in the artery corresponds to the size of the catheter or percutaneous introducer sheath used, which devices may typically range in diameter from 5 French (1.67 mm) for a diagnostic procedure to 6-10 French (2.00 mm-3.33 mm) for a therapeutic procedure. A number of techniques are known to facilitate closure and healing of the arteriotomy. One technique includes application of pressure at the puncture site for a relatively extended length of time. More particularly, compression has traditionally been applied to the puncture site for at least 30-45 minutes for the wound to close naturally after removal of the catheter. Patients are required to remain decumbent, essentially motionless and often with a heavy sandbag placed on their upper leg, for several hours to ensure that the bleeding has stopped. The recovery time from the medical procedure may be as little as half of an hour, but the recovery time from the wound can exceed 24 hours. This makes wound site management the longer critical care item. The longer the recovery time, the more expensive the procedure becomes, the greater the patient discomfort, and the greater the risk of complications. Other approaches to arteriotomy closure include a compression clamp device, a thrombotic or collagen plug, biological adhesives adapted to seal the arteriotomy, and/or suturing devices.

In addition, medical stapling systems have been proposed to facilitate closure and heating of the arteriotomy and resolve some of the concerns associated with arteriotomy closure after vascular catheterization procedures. Staples having four staple legs have proved very effective in holding the arteriotomy together. However, delivery of a four-legged staple is difficult. Percutaneous catheterization of blood vessels is performed at a non-orthogonal approach angle to prevent injury to the vessel or kinking of instruments and to avoid patient discomfort. Thus, the tissue track and the blood vessel form relatively shallow included angle. Due to the fact that the stapler typically encounters the artery at an angle, a first pair of staple legs often encounters the vessel tissue before the remaining second pair of staple legs. When the first pair of staple legs encounters vessel tissue, the staple is expanded. However, due to the angle between the staple and the artery, the second pair of staple legs may be deployed and subsequently closed within the lumen of the arteriotomy rather than engaging the vessel tissue adjacent the arteriotomy. Thus, the angle between the staple and the artery may cause the second pair of staple legs to “miss” the vessel tissue surrounding the arteriotomy when the staple is closed. To address this problem, clinicians are trained to distort the tissue track by raising the stapling system towards a more orthogonal angle. However, this technique is not always effective because the clinician cannot directly view the arteriotomy that is being closed. Therefore, it is desirable to provide an arteriotomy stapling system that compensates and adjusts for the angle of the artery so that all four staple legs may encounter the vessel tissue surrounding the arteriotomy substantially simultaneously.

### BRIEF SUMMARY OF THE INVENTION

Embodiments of the present invention are related to a stapling system for closing an arteriotomy. The stapling system includes a tissue staple and a stapler for delivering the staple to the arteriotomy. The tissue staple includes a pair of clips, each clip having two parallel legs connected by a base and terminating in pointed tips, wherein the clips face each other and are interconnected by two spaced-apart bands extending between the bases. The staple has a static configuration symmetrical about an imaginary axis extending between the clips. In the static configuration, the tips of each clip are closer to the axis than the base of the corresponding clip. The stapler is adapted to temporarily deform the staple from the static configuration into a delivery configuration in which the clips are parallel to each other and the bands of the staple are slanted with respect to the axis such that the tips of one clip are axially offset from the tips of the opposing clip. After the staple tips are embedded in the vessel wall around the arteriotomy, the staple is released from the stapler whereupon the staple tends to resiliently return to the static configuration.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is an isometric illustration of a staple in accordance with the invention, the staple shown in a static configuration.

FIG. 2 is a side view of the staple of FIG. 1.

FIG. 3 is an isometric view of the staple of FIG. 1 in a delivery configuration.

FIG. 4 is a side view of the staple of FIG. 3.

FIG. 5 is an isometric view of a stapling system in accordance with an embodiment of the present invention.

FIG. 6 is an isometric view of an expander of the stapling system shown in FIG. 5.

FIG. 7 is an isometric view of a retainer of the stapling system shown in FIG. 5.

FIG. 8 is a side view of a stapling system for delivering a staple in a delivery configuration.

FIG. 8A is a longitudinal sectional view of the stapling system for delivering a staple in a delivery configuration taken along line A-A of FIG. 8.

FIG. 9 is a top view of a stapling system for delivering a staple in a delivery configuration.

FIG. 9A is a longitudinal sectional view of the stapling system for delivering a staple in a delivery configuration taken along line A-A of FIG. 9.

FIGS. 10-16 illustrate a method for closing an arteriotomy in accordance with an embodiment of the present invention.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful. Furthermore, there is no intention to be bound by any expressed or implied theory presented in the preceding technical field, background, brief summary or the following detailed description.

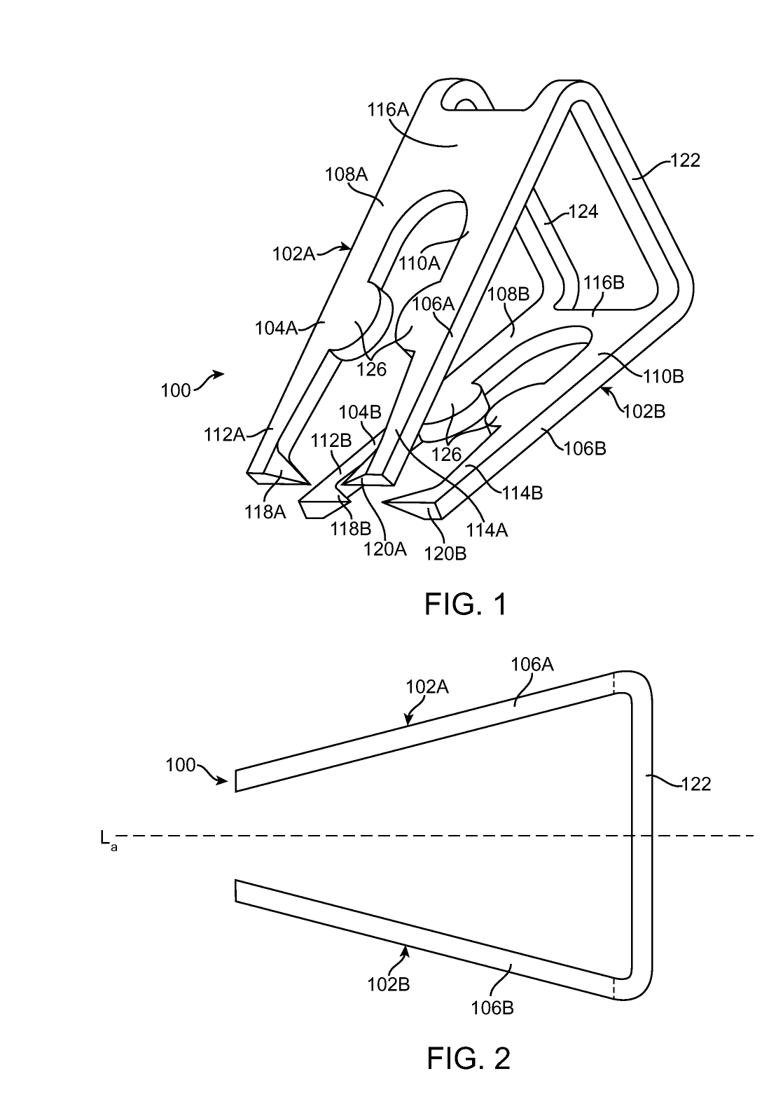

FIGS. 1-4 illustrate a staple 100 in accordance with an embodiment of the present invention. Staple 100 is provided to close a vascular puncture following a procedure. However, it should be apparent to those of ordinary skill in the art that staple 100 is not just limited to vascular repair but may be used for general tissue repair. Staple 100 has a resilient body including a pair of interconnected clips that face each other, a first clip 102A and a second clip 102B. First clip 102A and second clip 102B are connected by two spaced-apart bands, a first band 122 and a second band 124, extending between the proximal portions of the clips. First clip 102A includes a first leg 104A and a second leg 106A. First leg 104A and second leg 106A are generally parallel to each other. First leg 104A and second leg 106A include proximal portions 108A, 110A and distal portions 112A, 114A, respectively. Proximal portions 108A and 110A of first leg 104A and second leg 106A are connected by a base 116A. Distal portions 112A and 114A of first leg 104A and second leg 106A terminate in spicules or pointed tips 118A and 120A, respectively, which extend inwardly in a transverse direction towards each other. Similarly, second clip 102B includes a first leg 104B and a second leg 106B. First leg 104B and second leg 106B are generally parallel to each other. First leg 104B and second leg 106B include proximal portions 108B, 110B and distal portions 112B, 114B, respectively. Proximal portions 108B and 110B of first leg 104B and second leg 106B are connected by a base 116B. Distal portions 112B and 114B of first leg 104B and second leg 106B terminate in spicules or pointed tips 118B and 120B, respectively, which extend inwardly in a transverse direction towards each other. Pointed tips 118A, 120A, 118B, and 120B are provided to pierce the vessel tissue about the arteriotomy such that staple 100 securely grasps or clutches the tissue.

Each leg of each clip includes a rounded protrusion 126 for contacting an expansion mechanism of a stapler, as will be explained in further detail below. Protrusions 126 are located along the length of each leg, and are positioned spaced apart from the distal ends of the staple legs. Protrusions 126 are generally defined by a relatively flat portion extending inwardly in a transverse direction relative to an imaginary axis Lₐ such that the protrusions on legs 104A, 104B extend towards the protrusions on legs 106A, 106B, respectively, and vice versa. In the embodiment shown in FIG. 1, protrusions 126 are shown approximately in the middle of the length of each leg. On each clip 102A, 102B, protrusion 126 may extend transversely to contact an opposing protrusion or to form a gap therebetween, as shown. The transverse dimensions and position of protrusion 126 along the length of each leg may be selected, in conjunction with the dimensions of the expander mechanism, to facilitate the extent to which the pointed tips are separated during deployment of the staple, as will be described in further detail below.

FIGS. 1 and 2 illustrate staple 100 in a static configuration with no forces applied thereto to cause deformation. Staple 100 is formed in the static configuration and resiliently returns to the static configuration after deformation forces applied to the staple are removed. Thus, when released from a stapler, as explained in further detail below, staple 100 closes itself around the arteriotomy. As apparent from the side view of FIG. 2, staple 100 in the static configuration is symmetrical about axis Lₐ that extends between first clip 102A and second clip 102B. In the static configuration, first and second bands 122, 124 interconnecting first and second clips 102A, 102B are generally orthogonal to axis Lₐ. The legs of each clip extend distally from bases 116A, 116B. The legs of each clip extend inwardly towards axis Lₐ such that the distal portion of each leg is closer to axis Lₐ than the proximal portion of the same leg. In one embodiment, clips 102A and 102B form an included angle of approximately forty-five degree angles. However, the static configuration illustrated in FIGS. 1-2 is exemplary, viz., clips 102A and 102B may form an included angle other than forty-five degree angles, and first and second bands 122, 124 may be disposed other than orthogonal to axis Lₐ.

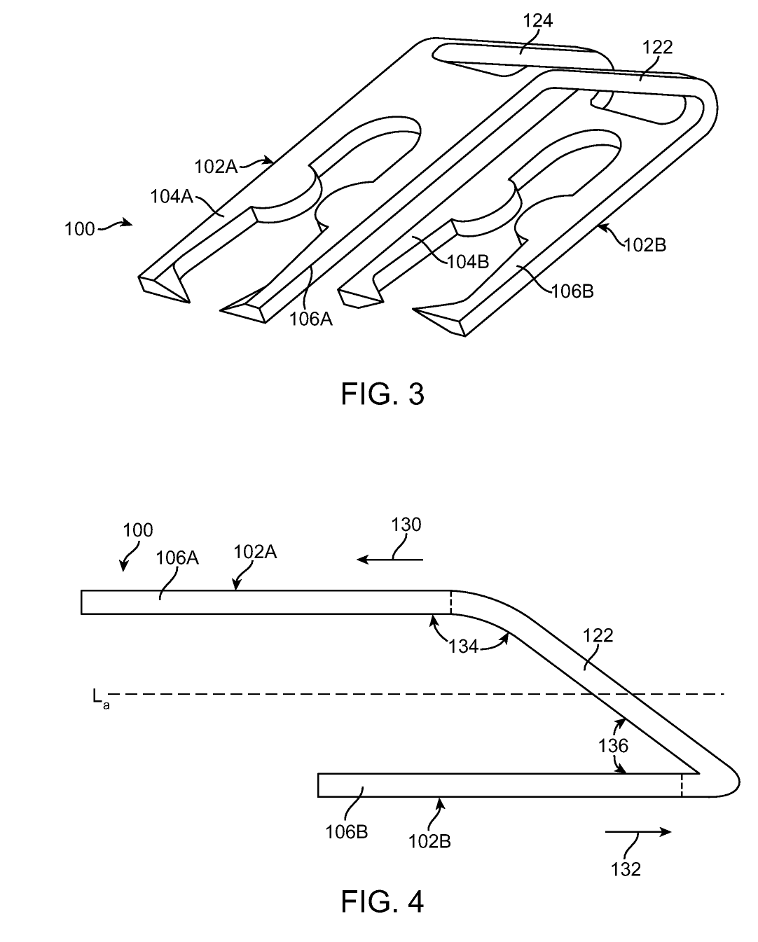

FIGS. 3 and 4 illustrate staple 100 in a delivery configuration, in which the offset relationship of the pointed tips of staple 100 accommodates a tissue track that is not orthogonal to the vessel wall and permits all of the pointed tips to pierce tissue about the arteriotomy substantially simultaneously. A stapler described in further detail below is adapted to temporarily deform staple 100 from the static configuration shown in FIGS. 1 and 2 into the delivery configuration shown in FIGS. 3 and 4. Upon release from the stapler, staple 100 tends to resiliently return from the delivery configuration shown in FIGS. 3 and 4 to the static configuration shown in FIGS. 1 and 2. In the delivery configuration, first and second clips 102A, 102B extend generally parallel to each other and first and second bands 122, 124 are oblique or slanted with respect to axis Lₐ.

The stapler deforms staple 100 into the delivery configuration such that first and second bands 122, 124 are shifted from their position orthogonal to axis Lₐ causing first clip 102A to be displaced in a distal direction indicated by directional arrow 130, and second clip 102B to be displaced in a proximal direction indicated by directional arrow 132. As a result, first clip 102A extends from bands 122, 124 of staple 100 at an angle 134 greater than ninety degrees, and second clip 102B extends from bands 122, 124 of staple 100 at an angle 136 less than ninety degrees. In an embodiment, angle 134 is approximately 135°, and angle 136 is approximately 45°.

Deformation that causes relative longitudinal displacement at the proximal portions of the clips creates corresponding relative longitudinal displacement at the distal portions of the clips. Thus, because clips 102A and 102B are of substantially equal length, the slanting of bands 122, 124 with respect to axis Lₐ causes the pointed tips of first clip 102A to be axially staggered or offset from the pointed tips of second clip 102B. Stated another way, the pointed tips of first clip 102A are located distally relative to the pointed tips of second clip 102B.

Staple 100 is formed from a resilient material. For example, staple 100 may be constructed out of a spring-type or superelastic material such as “beta” titanium (15-3-3-3 spring stock), nickel-titanium (nitinol), a nickel-tin alloy, a shape memory material, and other superelastic materials. In one embodiment, staple 100 may be formed from a bioabsorbable and/or biodegradable material that absorbs or degrades in vivo over time.

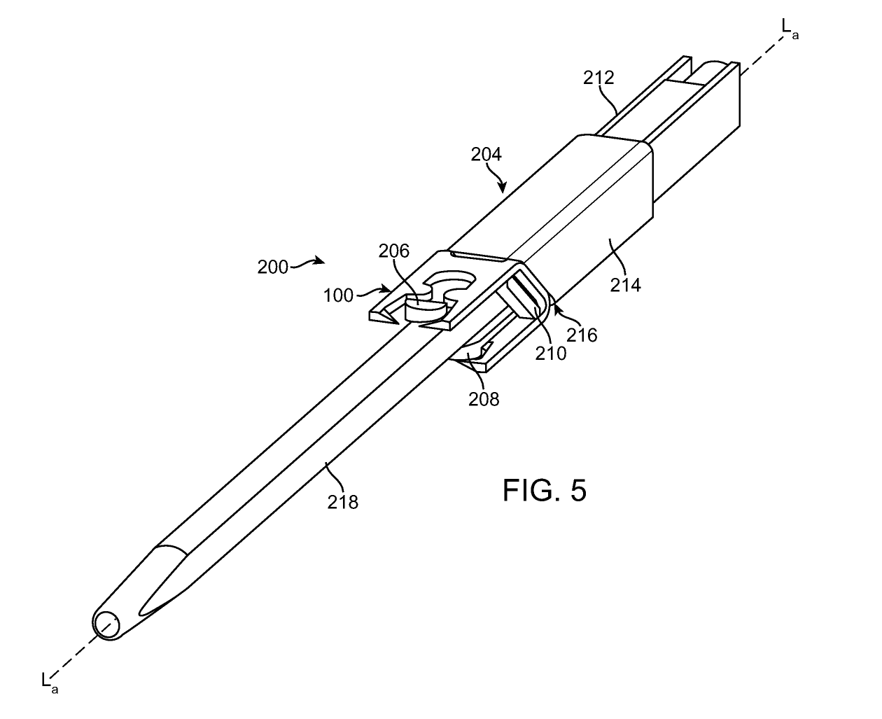

FIG. 5 is an isometric view of a stapling system 200 in accordance with an embodiment of the present invention. Stapling system 200 includes a stapler 204 for delivering staple 100 to an arterial puncture or arteriotomy. Imaginary axis Lₐ extends through the center of system 200 in a longitudinal direction. Stapler 204 includes an elongate sleeve 214 including an oblique distal tip 216 for holding staple 100. Stapler 204 includes actuator mechanisms for deforming staple 100 into a delivery configuration and for causing the legs of staple 100 to splay or expand outwardly in a transverse direction such that staple 100 may pierce tissue surrounding the arteriotomy.

The first actuator mechanism is a retention system for deforming first and second bands 122, 124 of staple 100 into a slanted angle with respect to axis Lₐ of system 200, as shown in the delivery configuration of FIGS. 3 and 4, and for holding first and second bands 122, 124 in the slanted angle against oblique distal tip 216 of sleeve 214. The retention system includes two elongate retainers, a first retainer 210 and a second retainer 212. Retainers 210, 212 are slidable within sleeve 214 in an axial direction beside a dilator 218 and extend to the proximal portion of the system (not shown) where they may be controlled to retain and subsequently release the staple at the distal portion of the system. Retainers 210, 212 may be constructed out of any appropriate biocompatible material, such as stainless steel. FIG. 7 illustrates an isometric view of first retainer 210 removed from system 200. It will be understood that the features shown and described with respect to first retainer 210 are also present in mirror-image form on second retainer 212. First retainer 210 includes a proximal portion 217, a distal end 215, and a shaft 213 that extends therebetween. Shaft 213 may be a generally flat strip of material having a thickness T1. Retainer 210 includes a protruding latch portion 219 at distal end 215 which has a thickness T2 that is greater than thickness T1.

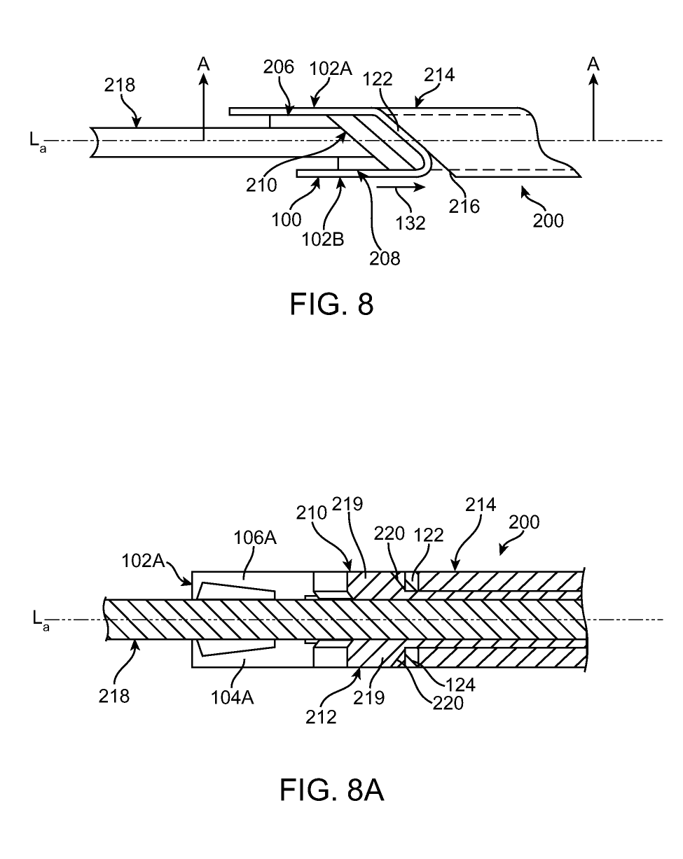

Latch portion 219 is sized and shaped to mate with either first or second band 122 or 124 on the distally-facing surface thereof. As such, latch portion 219 may comprise a rectangular solid or a plurality of hooks (not shown). As shown in FIGS. 5 and 8A, expanders 206, 208 and dilator 218 span and maintain the space between retainers 210, 212, thus supporting the engagement of latch portions 219 with bands 122, 124. As will be described further below, expanders 206, 208 and dilator 218 are withdrawn from the region of staple 100 in order to permit latch portions 219 to move inwardly and disengage from staple 100. Latch portion 219 is disposed at an angle to shaft 213 suitable for deforming first and second bands 122, 124 of staple 100 into a slanted angle with respect to axis Lₐ of system 200, as shown in the delivery configuration of FIGS. 3 and 4. In an embodiment, latch portion 219 extends at approximately a forty-five degree angle with respect to shaft 213 and axis Lₐ of system 200.

FIGS. 8 and 8A illustrate first and second retainers 210, 212 holding staple 100 in the delivery configuration against oblique distal tip 216 of sleeve 214. FIG. 8 is a side view of stapling system 200 and FIG. 8A is a longitudinal sectional view taken along line A-A of FIG. 8. Latch portions 219 of retainers 210, 212 engage first and second bands 122, 124 at the proximal portion of staple 100, respectively, to deform and retain the staple in the delivery configuration. When engaged with the retainers, first and second bands 122, 124 of staple 100 are deformed against oblique distal tip 216 of sleeve 214 to extend at the same angle as latch portions 219 and oblique distal tip 216.

During loading of staple 100 onto stapler 204, first and second retainers 210, 212 are slid proximally within sleeve 214 such that latches 219 engage first and second bands 122, 124. The proximal end of clip 102A is first brought into abutment against sleeve distal tip 216; then first and second bands 122, 124 are deformed into abutment flush against oblique distal tip 216 such that clip 102B is longitudinally displaced in a proximal direction as indicated by directional arrow 132. As described above, because clips 102A and 102B are of substantially equal length, the slanting of bands 122, 124 with respect to axis Lₐ causes the pointed tips of first clip 102A to be axially staggered or offset from the pointed tips of second clip 102B.

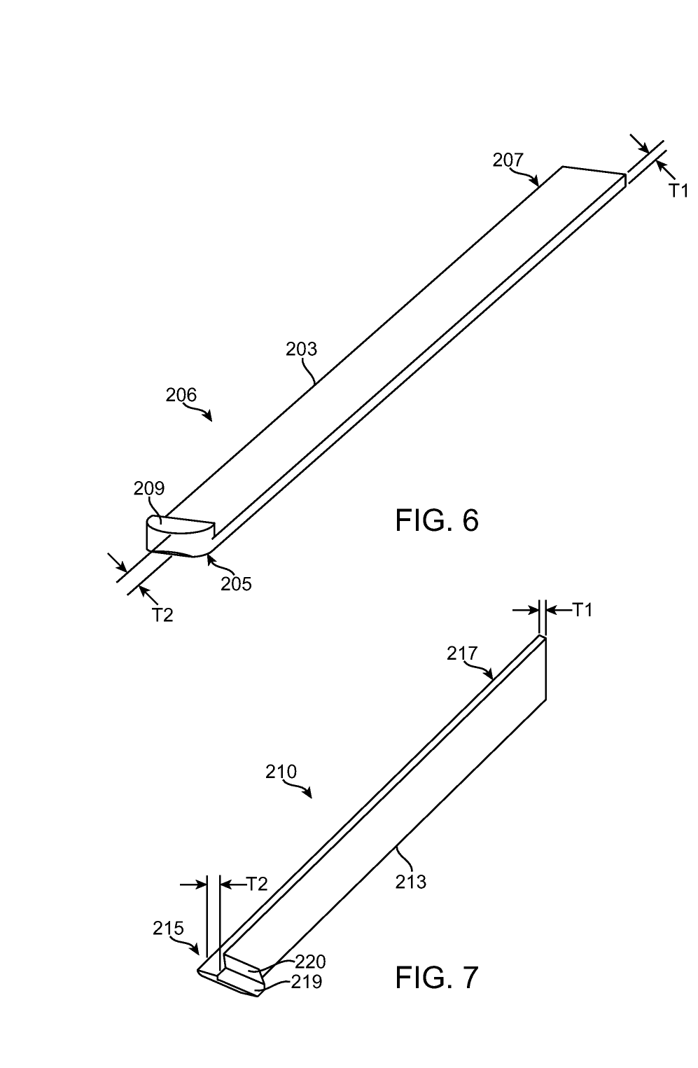

Referring back to FIG. 5, the second actuator mechanism is an expander mechanism for expanding the legs of staple 100 outwardly in a transverse direction. The expander mechanism also serves to hold clips 102A and 102B parallel in the delivery configuration. The expander mechanism includes two elongate expanders: a first expander 206 to expand legs 104A and 106A of first clip 102A of staple 100 and a second expander 208 to expand legs 104B and 106B of second clip 102B of staple 100. Expanders 206, 208 are slidable within sleeve 214 in an axial direction beside dilator 218 and extend to the proximal portion of the system where they may be controlled to expand the staple legs at the distal portion of the system. Expanders 206, 208 may be constructed out of any appropriate biocompatible material, such as stainless steel. FIG. 6 illustrates an isometric view of first expander 206 removed from system 200. It will be understood that the features shown and described with respect to first expander 206 are also present in mirror-image form on second expander 208. First expander 206 includes a proximal portion 207, a distal end 205, and a shaft 203 that extends therebetween. Shaft 203 may be a generally flat strip of material having thickness T1. Expanders 206, 208 extend distally beyond distal tip 216 of sleeve 214. Dilator 218 spans and maintains the space between the expanders such that the expanders 206, 208 hold clips 102A and 102B parallel in the delivery configuration. Expander 206 includes a protruding wedge portion 209 at distal end 205 that has a thickness T2 that is greater than thickness T1. Prior to deployment and release of staple 100 from stapler 204, expander 206 is positioned such that wedge portion 209 is located distal to protrusions 126. Wedge portion 209 is sized and shaped to force protrusions 126 apart as staple 100 and expanders 206, 208 are longitudinally displaced relative to each other. Wedge portion 209 may be circular, oval, or flared in shape.

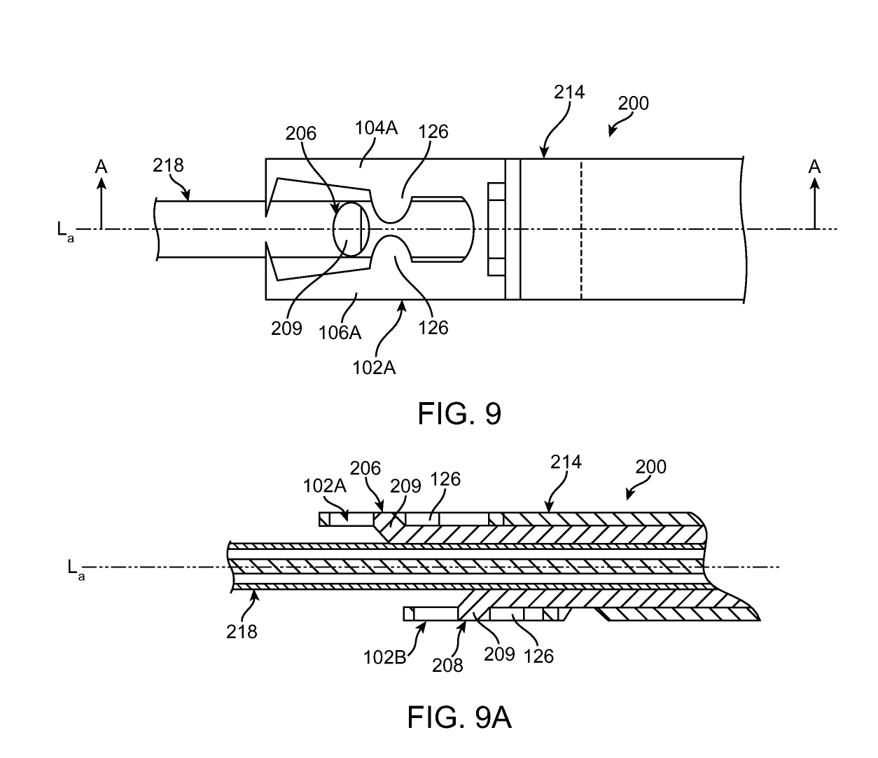

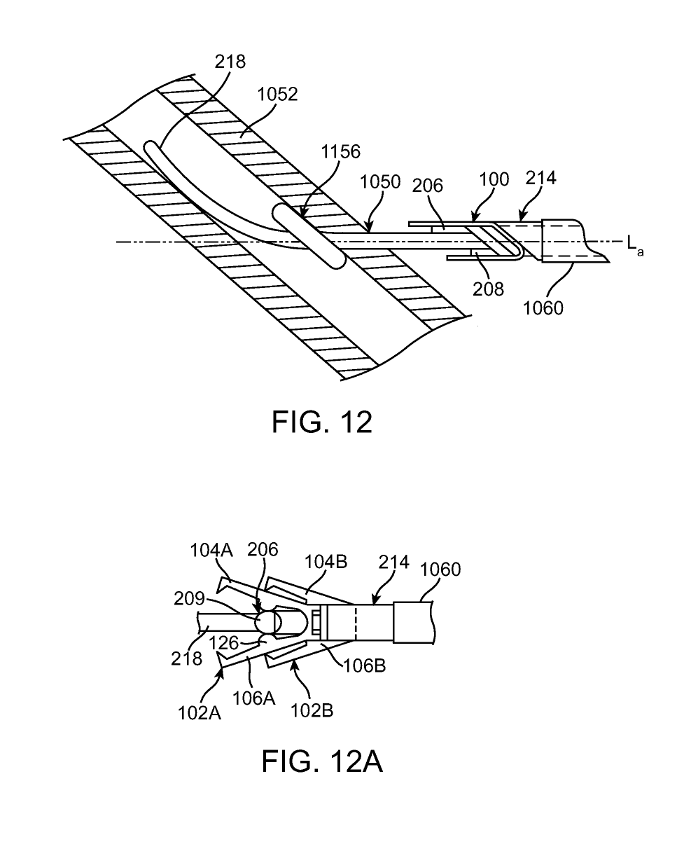

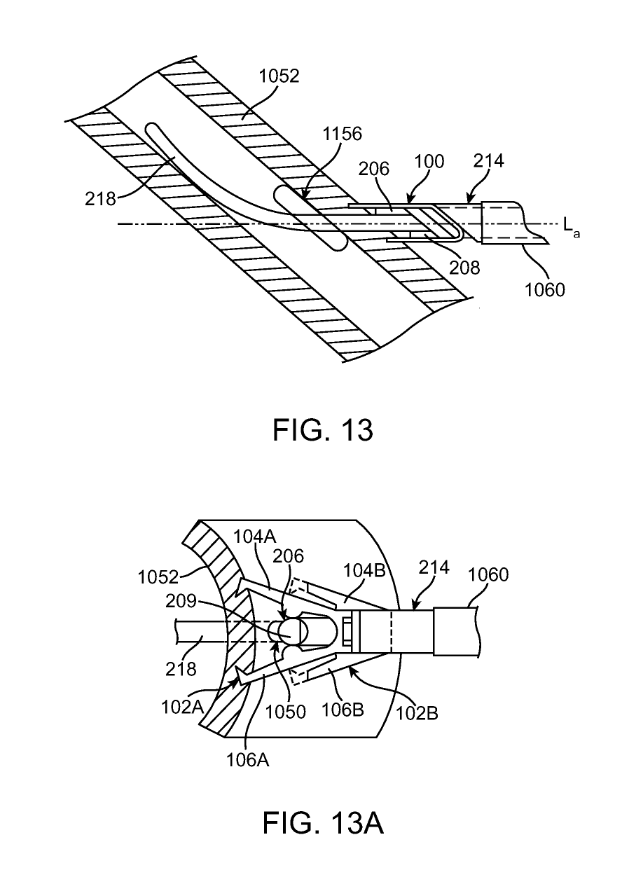

FIG. 9 is a top view of stapling system 200 and FIG. 9A is a sectional view taken along line A-A of FIG. 9. When it is desired to expand staple 200, expanders 206, 208 and sleeve 214 are longitudinally displaced relative to each other such that wedge portions 209 of expanders 206, 208 are forced between protrusions 126 located on the legs of each clip, thereby splaying the legs apart and transforming staple 100 into an expanded configuration. See FIG. 12A. Bases 116A, 116B hold together the respective proximal leg portions 108A, 110A and 108B, 110B such that pointed tips 118A, 120A and 118B, 120B are spread apart in the expanded configuration. Expanders 206, 208 may be retracted proximally while sleeve 214 is held in place or sleeve 214 may be advanced distally while expanders 206, 208 are held in place to cause the relative movement between staple 100 and expanders 206, 208, or a combination thereof. While in the expanded configuration, the pointed tips of staple 100 engage tissue surrounding the arteriotomy. See FIG. 13A.

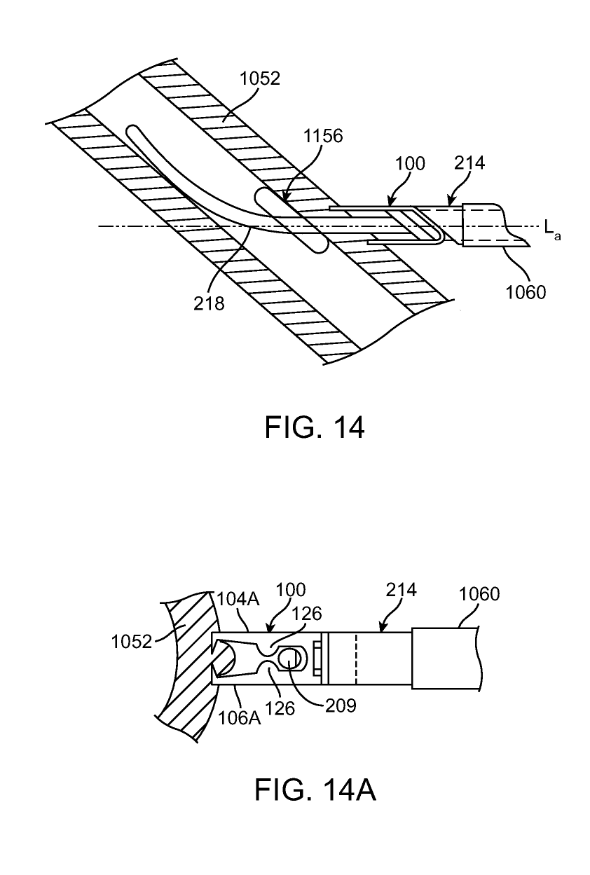

With tissue adjacent the arteriotomy engaged by the pointed tips of staple 100, expanders 206, 208 are further retracted until distal wedge portions 209 thereof are located proximal of protrusions 126, allowing the legs of each clip to move transversely toward one another such that each clip reverts toward the unexpanded configuration. See FIG. 14A. It is advantageous to extend the legs outwardly so that insertion of the pointed tips into the tissue occurs at locations separated by a greater distance than the distance separating the legs 104A, 106A and 104B, 106B when staple 100 is in the static configuration. Thus, the splayed legs can grasp a large portion of tissue around the wound site, thereby providing staple 200 with a more secure clutch on the tissue.

To release staple 100 from stapler 204, dilator 218 and expanders 206, 208 are withdrawn sufficiently to provide open space within staple 100 for latch portions 219 to deflect inwardly toward axis Lₐ and fit slidably between first and second bands 122, 124. The proximally-facing surfaces of latch portions 219 may be ramps 220 such that, when retainers 210, 212 are retracted in a proximal direction within sleeve 214, ramps 220 force latch portions 219 to disengage from first and second bands 122, 124 and to deflect inwardly toward axis Lₐ.

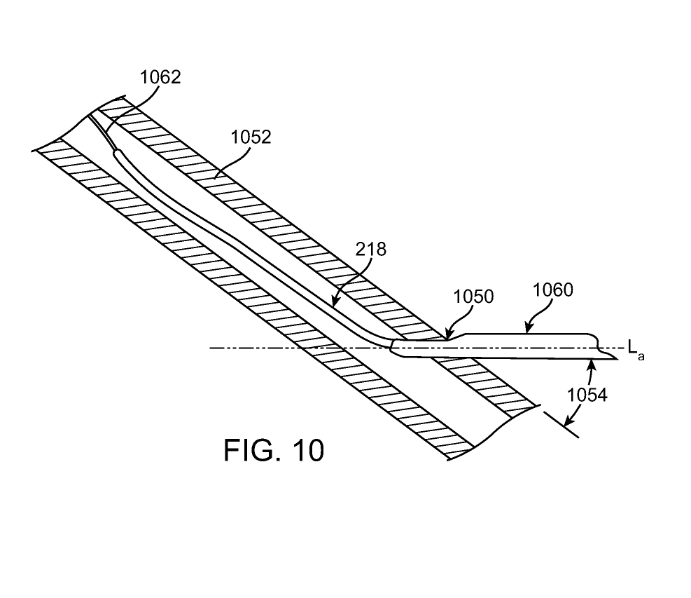

FIGS. 10-16 illustrate a method for closing an arteriotomy 1050. FIG. 10 illustrates the entry of dilator 218 into a vessel 1052, showing distal portion of the dilator 218 advanced along an indwelling guidewire 1062 in the region of an arteriotomy 1050. In an example, vessel 1052 may be an artery, which is typically reached during a catheterization procedure by creating a puncture wound that extends not only through the arterial wall, but also through a tissue track including various layers of tissue that are not shown for clarity. The tissue track extends through the patient's skin, subcutaneous and connective tissue, including fascia and the femoral sheath which are attached to the outer adventitia of vessel 1052. The tissue track typically encounters vessel 1052 at an angle 1054. Angle 1054 is usually between thirty-five and fifty-five degrees. Stapling system 200 of the present invention compensates and adjusts for angle 1054 so that all four legs of the staple encounter the vessel tissue surrounding arteriotomy 1050 substantially simultaneously.

A transition sheath 1060 is placed over dilator 218 for subsequent introduction of stapling system 200 therethrough. The distal end of dilator 218 may include a tapered tip portion to facilitate ingress through the skin and into vessel 1052. In an embodiment, dilator 218 may be part of stapling system 200 and have two or four flat sides to provide radial support and sliding abutment with expanders 206, 208 and retainers 210, 212. See FIG. 5. Alternatively, dilator 218 may have a round cross-section and be of a conventional type used with catheter introducer sheaths. In such an alternative embodiment, expanders 206, 208 and retainers 210, 212 may have curved inner surfaces to slidingly mate with and receive radial support from dilator 218.

Dilator 218 is a hollow elongated sheath and may have a blood marking lumen therethrough. When the practitioner urges the distal end of the dilator 218 into the vessel, the presence of fluid (blood) within the blood marking lumen indicates that the dilator 218 is properly positioned in vessel 1052. A blood marking inlet port (not shown) is located at a predetermined length along the dilator 218 to allow blood to flow into the blood marking lumen and spurt from the proximal end of stapling system 200 to alert the practitioner that dilator 218 is inserted in vessel 1052 to a desired depth, as will be understood by those familiar with the art.

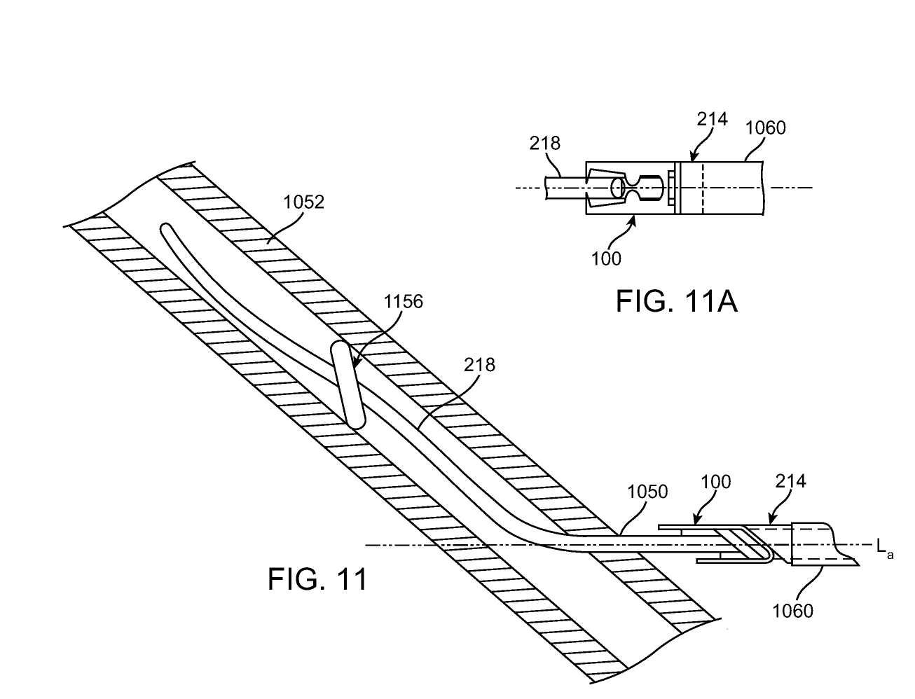

Once bloodmarking is observed, indicating that the dilator is properly positioned, a retention device 1156 located on dilator 218 is reversibly deployed into a larger transverse dimension, as shown in FIG. 11. Retention device 1156 may be an inflatable element or a mechanically actuated element for temporarily anchoring stapling system 200 in the desired position with respect to the vessel wall, as will be understood by those familiar with the art. Once retention device 1156 is deployed, it is pulled back or retracted until the clinician detects by tactile sensation that retention device 1156 abuts the inner wall of vessel 1052 as depicted in FIG. 12.

Transition sheath 1060 is pulled back and stapler 204 carrying staple 100 at its distal end is advanced within transition sheath 1060 to the region of arteriotomy 1050. Staple 100 is advanced to the target site in the delivery configuration. As can be seen in the top view illustrated in FIG. 11A, staple 100 is unexpanded as it is delivered to the site of arteriotomy 1050. Stapler 204 is operated to translate expanders 206, 208 relative to staple 100, thus transforming staple 100 into an expanded configuration proximal to arteriotomy 1050 wherein the legs of each clip 102A, 102B are expanded and the pointed tips thereof are separated from each other, as shown in the top view illustrated in FIG. 12A. As described above, legs 104A, 106A and 104B, 106B are expanded by relative movement between expanders 206, 208 and sleeve 214 such that distal ends 209 of expanders 206, 208 force apart protrusions 126, thereby pushing the legs apart. Although the legs are in the expanded configuration, staple 100 appears to be in the delivery configuration when viewed from the side, as depicted in FIG. 12.

As shown in FIGS. 13 and 13A, after the legs of each clip 102A, 102B are expanded, staple 100 is advanced in a distal direction to position the legs to clutch tissue of vessel 1052 surrounding the arteriotomy 1050. Alternatively, expanders 206, 208 may be held stationary with respect to the blood vessel while sleeve 214 is advanced distally thereover, causing simultaneous expansion of the staple legs and movement of the staple towards the vessel wall. Staple 100 is advanced against the vessel wall, causing the pointed tips of the staple 100 to contact or at least partially pierce tissue of vessel 1052. Although the legs of staple 100 are in the expanded configuration, staple 100 appears to be in the delivery configuration when viewed from the side, as depicted in FIG. 13.

With reference now to FIGS. 14 and 14A, expanders 206, 208 are withdrawn further proximally such that distal ends 209 of expanders 206, 208 clear protrusions 126, thereby allowing staple legs 104A, 106A and 104B, 106B to elastically recover and move transversely toward one another, as shown in top view FIG. 14A. Expanders 206, 208 may be withdrawn completely from staple 100 and further proximally away from sleeve distal end 216. As the staple legs revert toward the unexpanded delivery configuration, the tissue adjacent arteriotomy 1050, engaged by the pointed tips of staple 102, is drawn together.

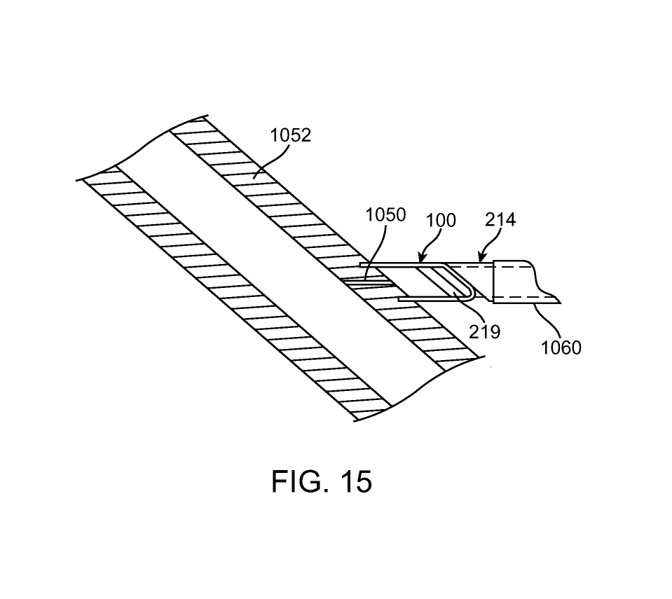

After the staple legs pierce and clutch tissue adjacent arteriotomy 1050, retention device 1156 is un-deployed and dilator 218 with retention device 1156 located thereon may then be removed, as shown in FIG. 15. Staple 102 remains in the delivery configuration as retention device 1156 is un-deployed and removed. Staple 100 is released from stapler 204 by retracting retainers 210, 212 proximally within sleeve 214. First, expanders 206, 208 and dilator 218 are withdrawn sufficiently to provide open space within staple 100 for latch portions 219 to deflect inwardly toward axis Lₐ and fit slidably between first and second bands 122, 124. When first retainers 210, 212 are retracted, ramps 220 force latch portions 219 to disengage from first and second bands 122, 124 and to deflect inwardly toward axis Lₐ.

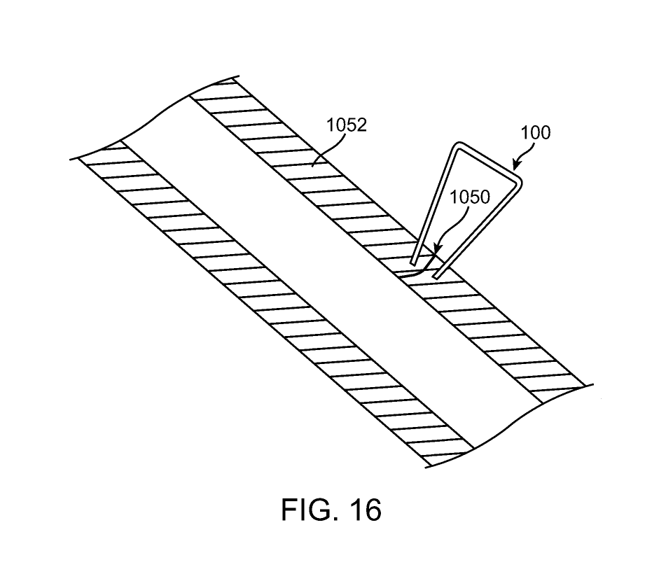

Referring now to FIG. 16, once retainers 210, 212 are withdrawn, staple 100 tends to revert back to the static configuration due to the static memory shape of staple 100. Pointed staple tips 118A, 120A tend to move toward pointed staple tips 118B, 120B, respectively, thus closing the arteriotomy. As staple 100 reverts toward the static configuration, it tends to rotate approximately 35-45° and stand upright or approximately orthogonal to vessel 1052 because the pointed staple tips securely clutch the vessel wall and there are no stapling device elements disposed within staple 100 to keep it aligned with the angled tissue track. Stapler 204 is removed, leaving staple 100 in place to hold arteriotomy 1050 closed.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment.

What is claimed is:

1. An arteriotomy stapling system comprising:
a tissue staple including a pair of clips, each clip having two parallel legs connected by a base and terminating in pointed tips, wherein the clips face each other and are interconnected by two spaced-apart bands extending between the bases, the staple having a static configuration in which the staple is symmetrical about an imaginary axis extending between the clips and the bands of the staple are generally orthogonal with respect to the axis; and
a stapler adapted to deform the staple from the static configuration into a delivery configuration in which the clips are parallel to each other and the bands of the staple are slanted with respect to the axis such that the tips of one clip are axially offset from the tips of the opposing clip.

2. The stapling system of claim 1, wherein the tips of each clip are closer to the axis than the base of the corresponding clip when the staple is in the static configuration.

3. The stapling system of claim 1, wherein, when the staple is in the delivery configuration, a first clip of the pair of clips forms an angle with the two bands of greater than ninety degrees and a second clip of the pair of clips forms an angle with the bands of less than ninety degrees.

4. The stapling system of claim 3, wherein the angle greater than ninety degrees is approximately 135° and the angle less than ninety degrees is approximately 45°.

5. The stapling system of claim 1, wherein the stapler includes a first elongate expander for outwardly expanding the legs of a first clip of the pair of clips and a second elongate expander for outwardly expanding the legs of a second clip of the pair of clips.

6. The stapling system of claim 5, wherein the leg of each clip has located therealong a protrusion directed transversely toward an opposing protrusion on the same clip, and wherein the first and second expanders each include a wedge portion at a distal end thereof for separating the opposing protrusions located along the legs of corresponding first and second clips.

7. The stapling system of claim 5, wherein the first and second expanders are slidably received in an outer sleeve.

8. The stapling system of claim 1, wherein the stapler includes a pair of elongate retainers for deforming the staple into the delivery configuration, wherein the retainers each include a latch portion configured to engage a corresponding band of the staple and deform and retain the staple in the delivery configuration.

9. The stapling system of claim 8, wherein the retainers are slidably received in an outer sleeve.

10. The stapling system of claim 1, wherein the staple is formed from a resilient material such that the staple tends to revert back to the static configuration upon being released from the stapler.

11. The stapling system of claim 1, wherein each pointed tip is directed transversely toward an opposing tip on the same clip.

12. A method for closing an arteriotomy comprising:
providing a stapling system including a stapler for delivering a staple to the arteriotomy, the staple including a pair of clips, each clip having two parallel legs connected by a base and terminating in pointed tips, wherein the clips face each other and are interconnected by two spaced-apart bands extending between the bases, the staple having a static configuration in which the staple is symmetrical about an imaginary axis extending between the clips and the bands of the staple are generally orthogonal with respect to the axis;
positioning the staple on the stapler such that the stapler deforms the staple from the static configuration into a delivery configuration in which the clips are parallel to each other and the bands of the staple are oblique with respect to the axis such that the tips of one clip are axially offset from the tips of the opposing clip;
advancing the staple to the region of the arteriotomy in the delivery configuration;
expanding the legs of each clip with the stapler;
piercing tissue around the arteriotomy with the pointed tips of the expanded legs, wherein all legs of the staple encounter the tissue around the arteriotomy substantially simultaneously;
releasing the staple from the stapler such that the staple substantially reverts back to the static configuration and closes around the arteriotomy.

13. The method of claim 12, wherein the tips of each clip are closer to the axis than the base of the corresponding clip when the staple is in the static configuration.

14. The method of claim 12, wherein, when the staple is in the delivery configuration, a first clip of the pair of clips forms an angle with the two bands of greater than ninety degrees and a second clip of the pair of clips forms an angle with the bands of less than ninety degrees.

15. The method of claim 14, wherein the angle greater than ninety degrees is approximately 135° and the angle less than ninety degrees is approximately 45°.

16. The method of claim 12, wherein the step of expanding the legs of each clip with the stapler includes proximally withdrawing a first expander within an outer sleeve of the stapler in order to expand the legs of a first clip of the pair of clips and proximal withdrawing a second expander within the outer sleeve of the stapler in order to expand the legs of a second clip of the pair of clips.

17. The method of claim 12, wherein the leg of each clip has located therealong a protrusion directed transversely toward an opposing protrusion on the same clip, and wherein the first and second expanders each include a wedge portion at a distal end thereof for separating the opposing protrusions located along the legs of corresponding clips.

18. The method of claim 12, wherein the step of releasing the staple from the stapler includes proximally withdrawing a pair of retainers within an outer sleeve of the stapler.

19. The method of claim 12, wherein the staple is formed from a resilient material such that the staple tends to revert back to the static configuration when the staple is released from the stapler.

20. The method of claim 12, wherein each pointed tip is directed transversely toward an opposing tip on the same clip.
